(19) United States
(12) Patent Application Publication
(10) Pub. No.: US 2011/0213342 A1
(43) Pub. Date: Sep. 1, 2011

(54) Real-time Virtual Indicium Apparatus and Methods for Guiding an Implant into an Eye

(76) Inventors: Ashok Burton Tripathi, Santa Barbara, CA (US); David Bragg, Laguna Niguel, CA (US)

(21) Appl. No.: 12/714,322

(22) Filed: Feb. 26, 2010

(51) Int. Cl. A61M 5/00; A61B 5/05

(52) U.S. Cl. 604/541; 600/425

(57) ABSTRACT

Disclosed herein are apparatus and associated methods for guiding an implant to a desired angle, a desired depth, and/or a desired position in an eye. The apparatus used to guide an implant into an eye includes: one or more real-time, multidimensional visualization modules; one or more displays to present one or more real-time, multidimensional visualizations; one or more data processors configured to produce real-time, virtual surgical reference indicia including data for guiding an implant to a desired angle, a desired depth, and/or a desired position an eye; and one or more inserter for guiding an implant to a desired angle, a desired depth, and/or a desired position in the anterior chamber of an eye. The associated methods generally involve the steps required for guiding an implant into the eye using the apparatus.

### FIELD OF THE INVENTION

Embodiments disclosed herein relate to the field of ocular surgery, more particularly to ocular surgical procedures including open or unmagnified surgery and micro-surgery, such as glaucoma surgery, utilizing visual imaging systems and devices for guiding an implant into the eye.

### BACKGROUND

One method of addressing the high intra-ocular pressure associated with glaucoma is through surgery. Ocular surgery is highly patient specific, being dependent on specific features and dimensions that, in certain cases, may be significantly different from those of expected norms. As a result, surgeons must rely upon their individual experience and skills to adapt whatever surgical techniques they are practicing to the individual requirements as determined by each patient's unique ocular structural features and dimensions.

To date, this individualized surgical adaptation is often accomplished essentially through freehand and best guess techniques based upon a pre-surgery examination and evaluation of each individual's ocular region and specific ocular features. This pre-surgical examination may include preliminary measurements as well as the surgeon making reference markings directly on the patient's ocular tissues with a pen or other form of dye or ink marking.

Further complicating matters, ocular tissues are not conducive to pre-surgery reference markings or measurements. This is particularly true because most ocular tissues have wet surfaces diminishing the quality of reference markings. Even further still, many ocular surgeries involve internal physical structures that cannot be accessed for direct measurement or marking prior to surgery, and therefore, the pre-surgical markings on external surfaces must be visually translated onto the relevant internal structures. This translation often leads to undesirable post-surgical outcomes.

Additionally, pre-surgical rinsing, sterilization, or drug administration to the ocular tissues prior to or during surgery may dissolve, alter or even remove reference markings. Similarly, subsequent wiping and contact with fluids, including the patient's body fluids, during the surgical procedure may remove or distort any reference markings from the ocular region of interest. As a result, surgical reference markings may lose any practical effectiveness beyond the initial stages of the surgical procedure, and in and of themselves, are not accurate as they present broad lines to indicate, in some procedures, micro-sized incisions.

Traditionally, glaucoma surgery involves a trabeculectomy, whereby an ophthalmic surgeon makes a small incision into the sclera of the eye for the purpose of allowing fluid to drain out of the eye and hence lower the pressure in the anterior chamber over time. Because the incisions heal and close over time, implanted shunts and stents have been developed allowing an opening to remain patent.

Implanting a stent or shunt can create a direct bypass through the trabecular meshwork and into Schlemm's canal resulting in increased aqueous outflow. Generally, the shunt or stent needs to be placed into the iridocorneal angle of the eye's anterior chamber, an area that cannot be easily viewed through the cornea by a surgeon using a microscope. As a result, gonioscopes, gonioprisms, or goniolenses have been utilized to see into the anterior chamber. Optical distortion is caused by the prism or mirror of a these devices, however, and surgeons have difficulty controlling the placement of the shunt while using a gonioscope.

Accordingly, in spite of the ongoing development and the growing sophistication of contemporary ocular surgery, there remains a need for improvement in the visualization of the anterior chamber of an eye and the provision of effective reference indicia including data for guiding an implant to a desired angle, depth, and position in an eye.

### SUMMARY

The exemplary embodiments of the apparatus systems and associated methods described herein provide for functional, useful, and effective ocular surgery reference markings, or indicia, including data and/or information for guiding an implant to a desired angle, a desired depth, and/or a desired position within the anterior chamber of an eye, and in one embodiment within the iridocorneal angle of the eye.

The apparatus or system for guiding an implant (such as a shunt, a stent, a drain, or a valve) into an anterior chamber of an eye described herein includes at least one real-time, multidimensional visualization module producing a real-time multidimensional visualization at least a portion of which is presented on at least one display. The system also includes: at least one data processor configured to produce at least one virtual indicium including data for guiding the at least one implant to a desired angle, a desired depth, and/or a desired position in said eye in conjunction with the at least one real-time multidimensional visualization and at least one inserter for guiding the implant into the eye in conjunction with the data processor and the real-time multidimensional visualization module. In one embodiment, the system may also include at least one stabilization ring configured to level, fixate, and/or orient the eye in conjunction with the data processor and the real-time multidimensional visualization module.

In some embodiments, the virtual indicia produced by at least one data processor include a real-time virtual stabilization ring. Moreover, in some embodiments, virtual indicia produced by at least one data processor include a real-time virtual implant. Further, in other embodiments virtual indicia produced by at least one data processor include a real-time virtual inserter tip.

In still other embodiments, at least one data processor includes an input for receiving pre-operative patient data to produce the data for guiding an implant to a desired angle, a desired depth, and/or a desired position. For example, pre-operative patient data can include at least one pre-operative stereoscopic still image. Another example of pre-operative patient data can include one or more optical coherence tomography (OCT) images. Pre-operative patient data may include at least one specific visual feature identifiable by a surgeon such as vasculature, vascular networks, vascular branching patterns, patterns or coloration in the iris, scratches on the cornea, dimples on the cornea, retinal features, the limbus, the pupillary boundary, deformities, voids, blotches, sequestered pigment cells, scars, darker regions, and combinations thereof.

In the systems described, the at least one real-time, multidimensional visualization can be three dimensional (3D) and/or in high definition (HD).

The stabilization ring described herein has at least one marking, which is identifiable by the at least one data processor. At least one data processor can utilize the at least one marking on the stabilization ring in calculating the position and/or level of said eye.

In embodiments where there is a stabilization ring that is not virtual, the stabilization ring can be sized to fit an eye or can be one size fits all. The stabilization ring can have either a substantially flat surface between the inner diameter and the outer diameter of a ring, or a raised surface between the inner and outer diameters. The at least one marking can be laser etched, painted, drawn, or molded on this substantially flat surface. Alternatively, the at least one marking can be made by light emitting diodes (LEDs) emitting either visible or non-visible wavelengths, such as, but not limited to, infrared LEDs. In one embodiment, the stabilization ring has at least one groove that directs the inserter into an eye and at least one marking that indicates the angle of such a groove. Further, in one embodiment, the stabilization ring has a handle attached for guiding, placing, and/or holding it on the eye. In other embodiments, the stabilization ring is disposable.

In accordance with the teachings of the present description, the at least one inserter has at least one marking identifiable by at least one data processor. The one data processor utilizes such a marking in calculating angle, depth, orientation, and/or position of the at least one inserter within the eye. In some embodiments, the inserter is disposable.

### BRIEF DESCRIPTION OF THE DRAWINGS

FIG. 1 is a cross-section of a human eye illustrating its structural elements and features.

FIG. 2 is an illustration of a gonioscope of the prior art being used in a glaucoma surgery.

FIG. 3 is an illustration of an exemplary image capture module of the present description.

FIG. 4 is an illustration of an exemplary apparatus of the present description retrofitted on a surgical microscope.

FIG. 5 is a schematic overview of an exemplary embodiment of an apparatus of the present description illustrating features thereof.

FIG. 6 is a plan view of an exemplary alignment control panel of the present description illustrating an exemplary embodiment of user input control thereof.

FIG. 7 is an illustration of an exemplary embodiment of a stabilization ring.

FIGS. 8A-E illustrate exemplary embodiments of an inserter and markings that can be made on an inserter.

FIG. 9 is a front view of a human eye illustrating specific visual features identifiable by a surgeon pre-operatively.

FIG. 10 is a front view an exemplary embodiment of a real-time 3D HD visualization of a human eye of a patient overlaid with an aligned HD pre-operative patient data still image of the patient eye.

FIG. 11 is a plan view of an exemplary embodiment of a stabilization ring and an exemplary embodiment of an inserter in use on an eye.

FIG. 12 is a cross-section of an eye with an exemplary embodiment of a stabilization ring placed on the eye's surface.

FIG. 13 is a front view of an exemplary embodiment of a real-time 3D HD visualization of an eye including a generated, real-time image of a virtual implant and virtual inserter tip.

FIG. 14 is a plan view of an exemplary embodiment of a generated real-time virtual stabilization ring on eye and an exemplary embodiment of an inserter used in reference to the virtual stabilization ring.

### DETAILED DESCRIPTION

Increased intra-ocular pressure associated with glaucoma can be addressed through surgery. Typically, glaucoma surgery involves a procedure whereby a small incision is made in the sclera of an eye in order to relieve the eye's internal pressure by allowing fluid to drain out of the anterior chamber of the eye. Such openings heal over time, resealing the anterior chamber of the eye where pressure can rebuild, and thus stents or shunts are implanted to maintain the openings and facilitate the drainage.

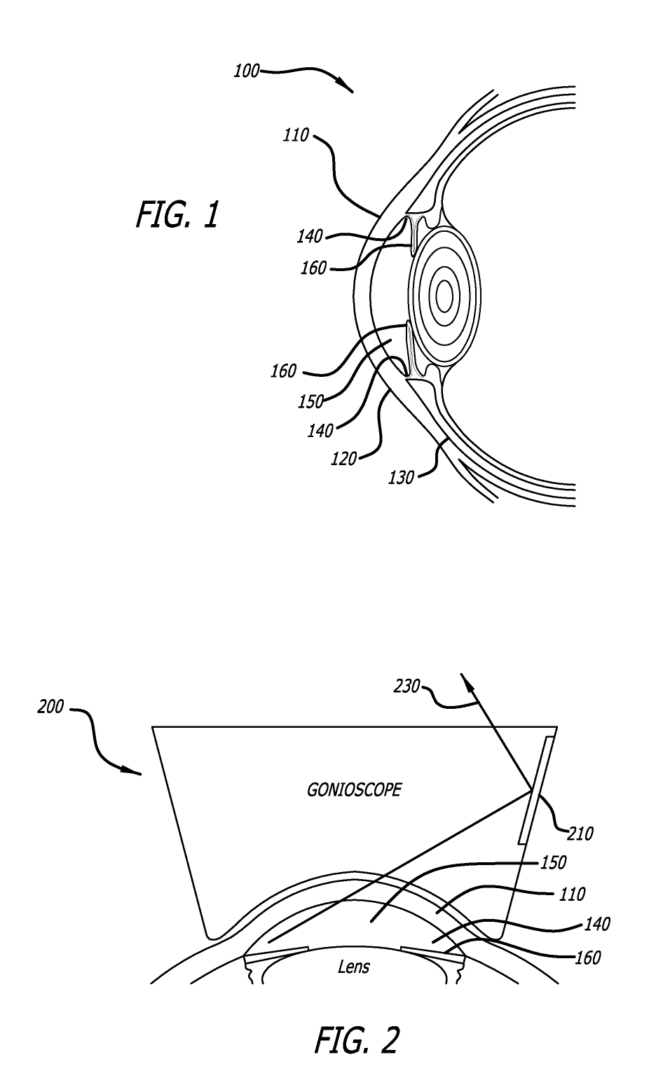

Such stents or shunts generally need to be placed into the iridocorneal angle in the anterior chamber of the eye (see FIG. 1), outside the line of sight of a patient. This is an area of the eye that cannot be easily viewed when using a stereomicroscope to see though the cornea. The iridocorneal angle is better viewed with a gonioscope, but because of optical distortion caused by the gonioscopes mirror and prism, surgeons have great difficulty in controlling accurate placement of a shunt while using a gonioscope (see FIG. 2).

Particularly, FIG. 2 illustrates a method of visualizing the anterior chamber for placement of an implant using an exemplary gonioscope of the prior art 200. Other prior art methods of visualizing the anterior chamber include using a goniolens or gonioprism (not shown). In this particular exemplary method, reflected image 230 of iridocorneal angle 140 or anterior chamber 150 between iris 160 and cornea 110 is reflected by gonioscope mirror 210. The use of gonioscope mirror 210 results in distortion of image 230. Such optical distortion in a surgeon's view of iridocorneal angle 140 or anterior chamber 150 makes accurate placement of an implant difficult, which is why there is room for improvement in the visualization and guidance of an implant into the anterior chamber.

Described herein are apparatus or systems and associated methods to provide clear navigational capability for a surgeon placing an implant into an eye. The exemplary embodiments are for generating one or more real-time, virtual indicium, or multiple indicia, including data for guiding an implant to a desired angle, a desired depth, and/or a desired position within the anterior chamber of an eye in conjunction with at least one real-time, multidimensional visualization of at least a portion of a target surgical field throughout a surgical procedure or any subpart thereof. In one exemplary embodiment, the apparatus and methods are for guiding an implant to a desired angle, a desired depth, and/or a desired position within the iridocorneal angle of the anterior chamber of an eye. The real-time, virtual indicia can include a virtual stabilization ring and/or a virtual implant and/or a virtual tip of the inserter that is used for guiding an implant into an eye.

In some embodiments, at least one element of the imaging described herein is stereoscopic. In one embodiment, the real-time, multidimensional visualization is stereoscopic three dimensional (3D) video and also may be in high definition (HD). Those of ordinary skill in the art will appreciate that a 3D HD real-time visualization will be most effective in enabling a physician to insert an implant in the anterior chamber, and in one embodiment within the iridocorneal angle of the anterior chamber of any eye. However, two dimensional (2D) systems or portions thereof can be useful according to the present description.

Moreover, the real-time, virtual indicia including data for guiding an implant to a desired angle, a desired depth, and/or a desired position within the anterior chamber of an eye can be placed under the direct control and adjustment of the operating surgeon or surgical team.

In a broad aspect, illustrating these beneficial features, an exemplary embodiment incorporates six primary elements: at least one real-time multidimensional visualization module, at least one display, at least one data processor with appropriate software which is configured to produce in real-time, one or more virtual indicium and/or generate display data on the real-time multidimensional visualization, at least one user control input, at least one virtual or actual stabilization ring, and at least one inserter. The elements of at least one real-time multidimensional visualization module, at least one data processor, at least one user control input can be physically combined into a single device or can be linked as physically separate elements within the scope and teachings of the present disclosure as required by the specific implant procedure being practiced.

An exemplary real-time multidimensional visualization module suitable for practicing the present methods incorporates the basic structural components of the Applicant's TrueVision Systems, Inc. real-time 3D HD visualization systems described in the Applicant's co-pending U.S. applications: Ser. No. 11/256,497 entitled “Stereoscopic Image Acquisition Device,” filed Oct. 21, 2005; Ser. No. 11/668,400 entitled “Stereoscopic Electronic Microscope Workstation,” filed Jan. 29, 2007; Ser. No. 11/668,420 entitled “Stereoscopic Electronic Microscope Workstation,” filed Jan. 29, 2007; Ser. No. 11/739,042 entitled “Stereoscopic Display Cart and System,” filed Apr. 23, 2007; Ser. No. 12/417,115, entitled “Apparatus and Methods for Performing Enhanced Visually Directed Procedures Under Low Ambient Light Conditions,” filed Apr. 2, 2009; Ser. No. 12/249,845, entitled “Real-time Surgical Reference Indicium Apparatus and Methods for Surgical Application,” filed Oct. 10, 2008; Ser. No. 12/390,388, entitled “Real-time Surgical Reference Indicium Apparatus and Methods for Intraocular Lens Implantation,” filed Feb. 20, 2009; Ser. No. 12/582,671, entitled “Real-time Surgical Reference Indicium Apparatus and Methods for Astigmatism Correction,” filed Oct. 20, 2009, all of which are fully incorporated herein by reference as if part of this specification.

The multidimensional visualization module is used to provide a surgeon with a real-time visualization of at least a portion of a target surgical field, which in the present application is an eye.

“Real-time” as used herein generally refers to the updating of information at essentially the same rate as the data is received. More specifically, “real-time” is intended to mean that the image data is acquired, processed, and transmitted from the photosensor of the visualization module at a high enough data rate and at a low enough time delay that when the data is displayed, objects presented in the visualization move smoothly without user-noticeable judder, latency or lag. Typically, this occurs when the processing of the video signal has no more than about 1/10^{th} second of delay.

It should be appreciated that while it is preferred to utilize a multidimensional visualization module that provides a surgeon with a real-time 3D visualization of at least a portion of the target surgical field, it is contemplated as being within the scope of the present disclosure for the visualization module to provide a real-time visualization that is a real-time 2D visualization. However, the use of a 3D visualization is preferred as it provides many benefits to the surgeon including more effective visualization and depth of field particularly with regard to the topography of an eye. In one embodiment, the visualization of the target surgical field is in high definition (HD).

The term “high definition” or “HD” as used herein can encompass a video signal having a resolution of at least 960 lines by 720 lines and to generally have a higher resolution than a standard definition (SD) video. For purposes of the present disclosure, this can be accomplished with display resolutions of 1280 lines by 720 lines (720p and 720i) or 1920 lines by 1080 lines (1080p or 1080i), or any resolution in between. In contrast, standard definition (SD) video typically has a resolution of 640 lines by 480 lines (480i or 480p) or less. It is however, within the scope of the present description that the multidimensional visualization can be in SD, though HD is preferred.

The exemplary embodiments of at least one real-time multidimensional visualization module, at least one data processor, at least one user control input described herein can be embodied in a single device which can be retrofitted onto existing surgical equipment such as surgical microscopes or open surgery apparatus or as a stand alone apparatus including its own optical systems. This is highly advantageous as retrofit embodiments can be added to existing systems, allowing expensive equipment to simply be upgraded as opposed to purchasing an entirely new system. The exemplary apparatus can include various optical or electronic magnification systems including stereomicroscopes or can function as open surgery apparatus utilizing cameras and overhead visualizations with or without magnification.

FIG. 1 is a cross-sectional view of a general structure of eye 100. Eye 100 includes cornea 110, the circumference of which is defined by limbus 120, which is the border between cornea 110 and sclera 130. Iridocorneal angle 140 in the anterior chamber 150 is the angle defined by iris 160 and cornea 110.

Using exemplary embodiments described herein, at least one implant including, but not limited to, a shunt, a stent, a drain, or a valve can inserted at a controlled and desired angle, depth, and/or position into anterior chamber 150 to facilitate drainage and relieve pressure from a an eye with pressure that is higher than normal and in one embodiment a glaucoma-diseased eye. In one embodiment, at least one implant can be inserted into iridocorneal angle 140 of anterior chamber 150.

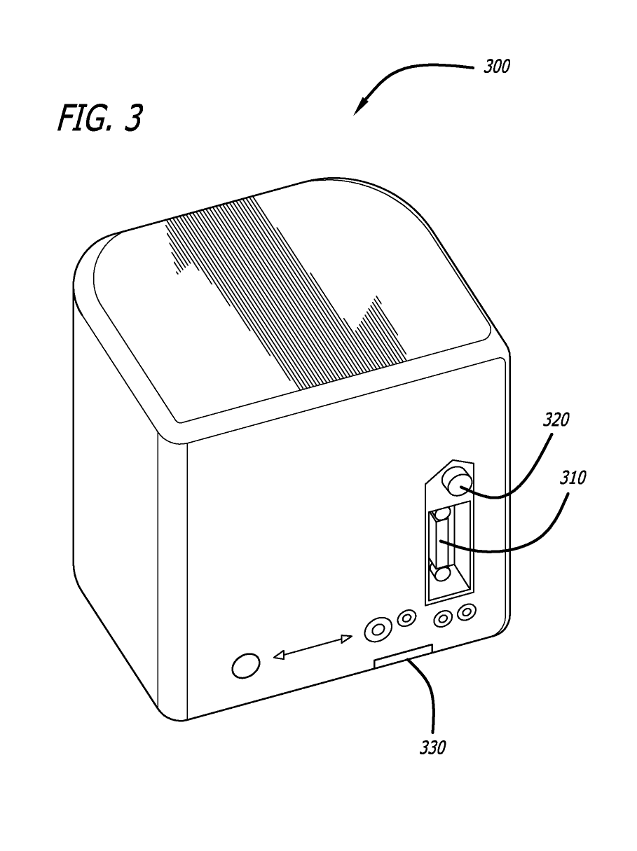

FIG. 3 illustrates image capture module 300 which includes a multidimensional visualization module and an image processing unit, both housed within image capture module 300, and therefore, not depicted. The exemplary image capture module comprises at least one photosensor to capture still images, photographs or videos. As those of ordinary skill in the art will appreciate, a photosensor is an electromagnetic device that responds to light and produces or converts light energy into an electrical signal which can be transmitted to a receiver for signal processing or other operations and ultimately read by an instrument or an observer. Communication with image capture module 300 including control thereof and display output from image capture module 300 are provided by first connector 310. Image capture module power is provided by second connector 320. Additionally, image capture module 300 can manually control the transmitted light intensity using iris slider switch 330.

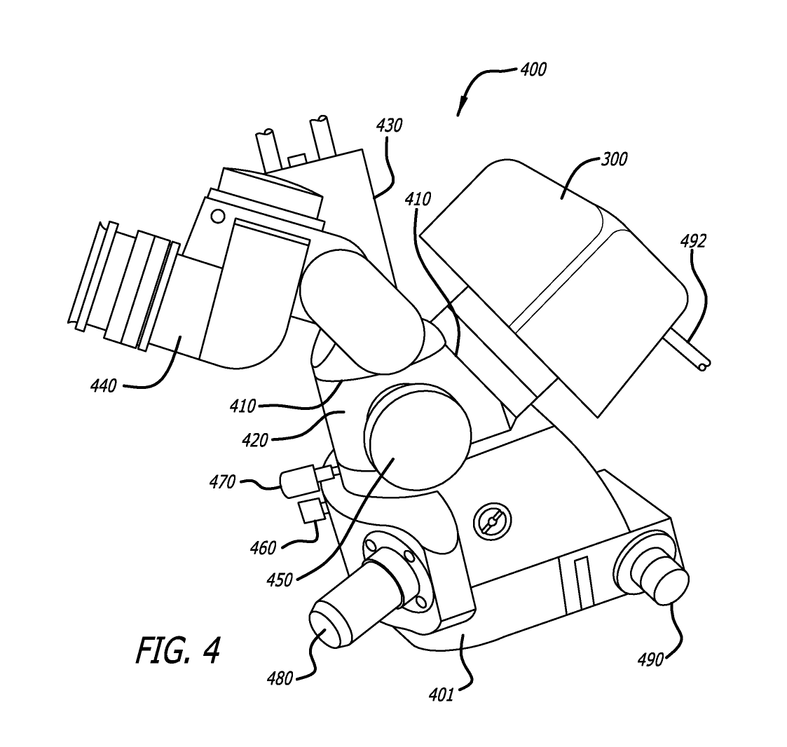

In another embodiment, FIG. 4 illustrates retrofitted surgical microscope 400 incorporating image capture module 300 retrofitted thereto. Retrofitted surgical microscope 400 includes image capture module 300 coupled to first ocular port 410 on ocular bridge 420. Further, ocular bridge 420 couples video camera 430 to a second ocular port (not shown) and binocular eyepiece 440 to third ocular port 410. Optional forth ocular port 450 is available for further additions to retrofitted surgical microscope 400. Although retrofitted surgical microscope 400 includes image capture module 300, it still retains the use of conventional controls and features such as, but not limited to, iris adjustment knob 460, first adjustment knob 470, second adjustment knob 480, illumination control knob 490, and an objective lens (not shown). Further still, image capture module 300 can send and receive information through signal cable 492 which is connected to first connector 310, while power is supplied via second connector 320 of image capture module 300.

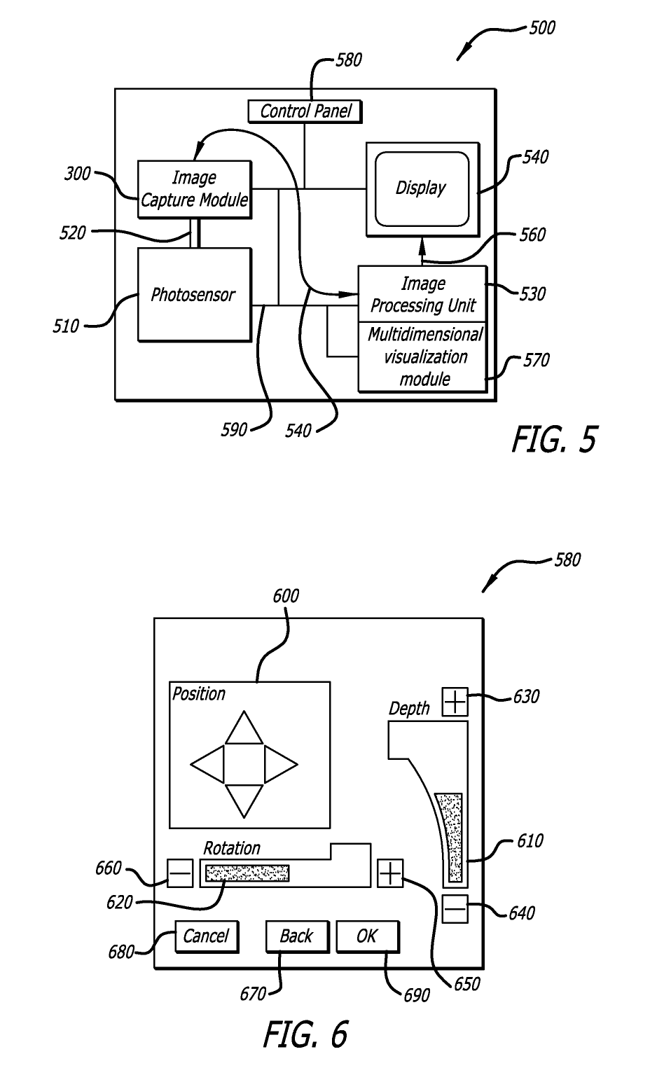

An exemplary, non-limiting configuration of components is illustrated in FIG. 5. Apparatus setup 500 includes image capture module 300, coupled to photosensor 510 by bi-directional link 520. Those of ordinary skill in the art will appreciate that bi-directional link 520 can be eliminated where image capture module 300 and photosensor 510 are physically the same device. Image capture module 300 is in direct communication with image processing unit 530 by first cable 540. First cable 540 can be a cable connecting to physically different devices, can be a cable connecting two physically different components within the same device, or can be eliminated if image capture module 300 and image processing unit 530 are physically the same device. First cable 540 allows, in certain embodiments, bi-directional communication between image capture module 300 and image processing unit 530. Image processing unit 530 generates images and videos that are displayable on display 540. It is within the scope of the present description that display 540 include multiple displays or display systems (e.g. projection displays). An electrical signal (e.g. video signal) is transmitted from image processing unit 530 to display 540 by a second cable 560, which is any kind of electrical signal cable commonly known in the art. Image processing unit 530 can be in direct communication with multidimensional visualization module 570, which can also send electrical signals to display 540 via second cable 560. In one embodiment, image capture module 300, image processing unit 530, and multidimensional visualization module 570 are all housed in a single device or are physically one single device. Further, one or all of the components of the present disclosure can be manipulated by control panel 580 via cable network 590. In one embodiment, control panel 580 is wireless.

“Display,” as used herein, for example display 540, can refer to any device capable of displaying a still or video image. Preferably, the displays of the present disclosure display HD still images and video images or videos which provide a surgeon with a greater level of detail than a SD signal. More preferably, the displays display such HD stills and images in stereoscopic 3D. Exemplary displays include HD monitors, cathode ray tubes, projection screens, liquid crystal displays, organic light emitting diode displays, plasma display panels, light emitting diodes, 3D equivalents thereof and the like. In some embodiments, 3D HD holographic display systems are considered to be within the scope of the present disclosure. In one embodiment, display 540 is a projection cart display system and incorporates the basic structural components of the Applicant's TrueVision Systems, Inc. stereoscopic image display cart described in the Applicant's co-pending U.S. application: Ser. No. 11/739,042. In another embodiment, display 540 is a high definition monitor, such as one or more liquid crystal displays (LCD) or plasma monitors, depicting a 3D HD picture or multiple 3D HD pictures.

The exemplary image processing units as illustrated in FIGS. 3, 4 and 5 include a microprocessor or computer configured to process data sent as electrical signals from image capture module 300 and to send the resulting processed information to display 540, which can include one or more visual displays for observation by a physician, surgeon or a surgical team. Image processing unit 530 may include control panel 580 having user operated controls that allow a surgeon to adjust the characteristics of the data from image capture module 300 such as the color, luminosity, contrast, brightness, or the like sent to the display.

In one embodiment, image capture module 300 includes a photosensor, such as a camera, capable of capturing a still image or video images, preferably in 3D and HD. However, the photosensor can also capture still images or video in 2D. It is within the teachings herein that the photosensor is capable of responding to any or all of the wavelengths of light that form the electromagnetic spectrum. Alternatively, the photosensor may be sensitive to a more restricted range of wavelengths including at least one wavelength of light outside of the wavelengths of visible light. “Visible light,” as used herein, refers to light having wavelengths corresponding to the visible spectrum, which is that portion of the electromagnetic spectrum where the light has a wavelength ranging from about 380 nanometers (nm) to about 750 nm.

More specifically, the at least one data processor can also be in direct communication with multidimensional visualization module 570 and/or image capture module 300. The data processors, in their basic form, are configured to generate display data based on information in the real-time visualization of at least a portion of the target surgical field produced by multidimensional visualization module 570 and/or produce at least one real-time virtual indicium including data for guiding said at least one implant to a desired angle, a desired depth, and/or a desired position in the anterior chamber of an eye in conjunction with the real-time visualization.

Non-limiting real-time, virtual indicia can include a real-time, virtual implant and/or a real-time, virtual tip of the inserter. In such embodiments, data processors will use the back half of an inserter as seen in the real-time visualization of the target surgical field to generate a real-time, virtual implant and/or a real-time, virtual tip of the inserter on the display. Such virtual indicia can allow a physician, a surgeon, or a surgical team to visualize the implant and/or inserter tip as it is inserted into the anterior chamber. Further, the at least one real-time virtual indicium produced by the data processors can include a real-time virtual stabilization ring to assist in the implant procedure.

The data processor or processors can be incorporated into multidimensional visualization module 570 or can be a stand alone processor such as a workstation, personal data assistant or the like. The at least one data processor is controlled by built-in firmware upgradeable software and at least one user control input, which is in communication with the data processors. The at least one user control input can be in the form of a keyboard, mouse, joystick, foot pedals, touch screen device, remote control, voice activated device, voice command device, or the like and allows the surgeon to have direct control over the one or more virtual surgical indicium and/or generated display data.

FIG. 6 illustrates an exemplary user control input, in the form of control panel 580. Control panel 580 includes multidirectional navigation pad 600 with user inputs allowing a controlling surgeon or operator to move data vertically, horizontally or any combination of the two. Additionally, the depth of the data can be adjusted using depth rocker 610 of control panel 580 and the rotation can be adjusted using rotation rocker 620 of control panel 580. Depth can be adjusted using both increase depth position 630 and decrease depth position 640 of depth rocker 610. Additionally, rotation can be adjusted using both increase rotation position 650 and decrease rotation position 660 of rotation rocker 620. Other non-limiting adjustments that can be made to the pre-operative image or to the real-time visualization include changes in diameter, opacity, color, horizontal and vertical size, and the like, as known to those of ordinary skill in the art. It should be noted that in exemplary control panel 580 an adjustment can be undone by the surgeon utilizing “back” button 670. Further, the entire process can be ended by the surgeon by engaging “cancel” button 680. Further, once the surgeon is satisfied with the alignment of the data, the alignment is locked into place by engaging “ok” button 690.

Alternative control panel embodiments for the manipulation and alignment of the pre-operative still image are contemplated as being within the scope and teachings of the present description. For example, a hand-held device such as a 3D mouse can be used as known in the art to directly position templates, images, and references within the real-time multidimensional visualization. Such devices can be placed on a tabletop or held in mid-air while operating. In another embodiment, foot switches or levers are used for these and similar purposes. Such alternative control devices allow a surgeon to manipulate the pre-operative data (including, but limited to, a still image), virtual indicia, and/or on-screen pointers without taking his or her eyes off of the visualization of a surgical procedure, enhancing performance and safety.

In yet another alternative embodiment, a voice activated control system is used in place of, or in conjunction with, control panel 580. Voice activation allows a surgeon to control the modification and alignment of the pre-operative data and its associated indicia as if he was talking to an assistant or a member of the surgical team. As those of ordinary skill in the art will appreciate, voice activated controls typically require a microphone and, optionally, a second data processor or software to interpret the oral voice commands. In yet a further alternative embodiment, a system is envisioned wherein the apparatus utilizes gesture commands to control pre-operative image adjustments. Typically, as known to those of ordinary skill in the art, the use of gesture commands involves an apparatus (not shown) having a camera to monitor and track the gestures of the controlling physician and, optionally, a second data processor or software to interpret the commands.

In one embodiment, apparatus setup 500 as illustrated in FIG. 5 can be used in many medical settings. For example, apparatus setup 500 can be used in an examination room. Therein, image capture module 300 utilizes photosensor 510 to capture pre-operative patient data such as still images, preferably in HD, and information relating to a patient's iridocorneal angle. Photosensor 510 can be coupled to any piece of medical equipment that is used in an examination room setting wherein pre-operative data can be captured. Image capture module 300 directs this data to image processing unit 530. Image processing unit 530 processes the data received from image capture module 300 and presents it on display 540.

In another embodiment, apparatus setup 500 can be used in an operating room. Therein, image capture module 300 utilizes photosensor 510 to capture a real-time visualization of at least a portion of the target surgical field, preferably in HD, more preferably in 3D. However, a 2D real-time visualization of at least a portion of the target surgical field is also possible. Image capture module 300 directs this data to image processing unit 530 including multidimensional visualization module 570. Image processing unit 530 including multidimensional visualization module 570 processes the data received from image capture module 300 and presents it on display 540 in real-time.

In still another embodiment, apparatus setup 500 is used in an operating room and photosensor 510 is a surgical microscope. Therein, image capture module 300 is retrofitted on the surgical microscope. The use of a surgical microscope in combination with apparatus setup 500 allows a surgeon to comfortably visualize a surgical procedure on one or more displays instead of staring for, in some cases, several hours though the eyepiece of a surgical microscope.

Apparatus setup 500 used in an examination room can be in direct communication with apparatus setup 500 used in the operating room. The two apparatus setups can be directly connected by cable, or indirectly connected through an intermediary device such as a computer server. In some embodiments, the two sections can be separate systems, even in different physical locations. Data can be transferred between the two systems by any means known to those of ordinary skill in the art such as an optical disc, a flash memory device, a solid state disk drive, a wired network connection, a wireless network connection or the like.

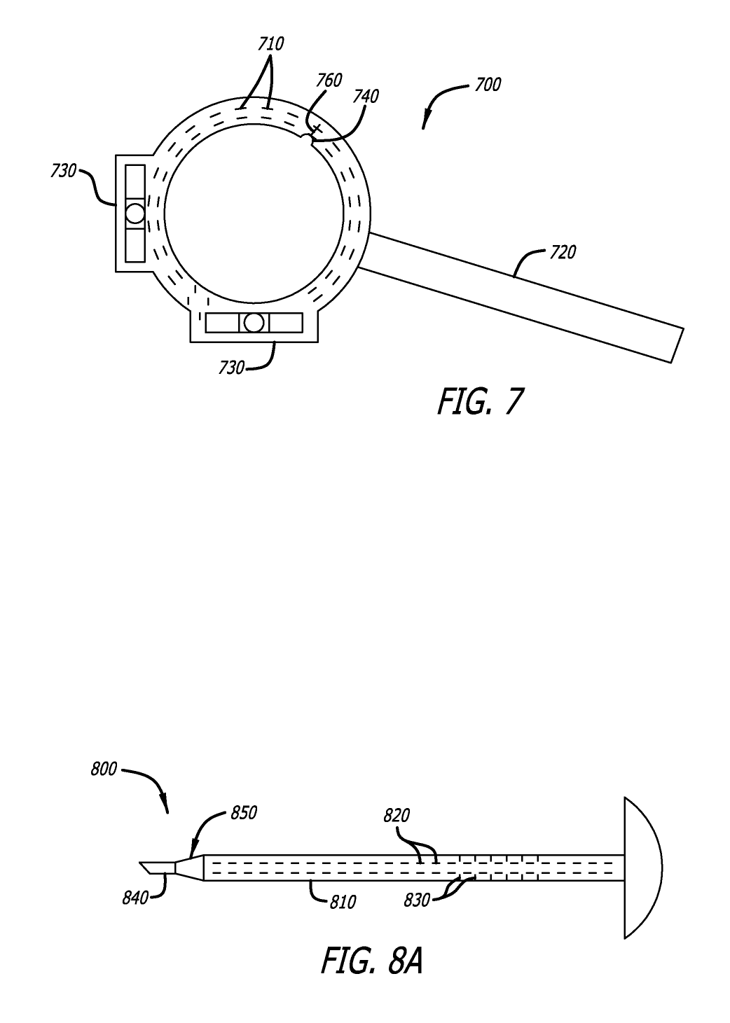

FIG. 7 is an illustration of an exemplary embodiment of stabilization ring 700. Stabilization ring 700 can be used by a surgeon to fixate, orient, and/or level an eye during ocular surgery. The surface between the inner and outer diameters of stabilization ring 700 can be substantially flat, raised, and/or curved. A substantially flat surface is the same or similar thickness or height between the underside and topside of stabilization ring 700 between the inner and outer diameters. A raised surface includes, but is not limited to, a rounded, a curvilinear, a concave, a convex, or a surface where the thickness or height between the underside and topside of stabilization ring 700 varies. In one embodiment, the thickness or height between the underside and topside increases gradually from the outer diameter to reach its maximum thickness or height midway between the inner and outer diameters and then decreases gradually to the inner diameter. A curved surface can be either a flat or raised surface where the underside of stabilization ring 700 is not on one plane. For example, a stabilization ring with a curved surface can include, but is not limited to, an embodiment where the underside of the stabilization is designed to fit to the curvature of an eyeball.

In one exemplary embodiment of stabilization ring 700, at least one marking 710 is laser etched, painted, drawn, molded along the surface of stabilization ring 700. Alternatively, at least one marking 710 can be indicated by LEDs emitting either visible or non-visible wavelengths, such as, but not limited to infrared LEDs. At least one marking 710 is designed to be identified by at least one data processor (not shown) when it appears in at least one real-time multidimensional visualization. In turn, in some embodiments, the at least one data processor (not shown) calculates the orientation and position of an eye during surgery and generates this data for display. In some embodiments, the at least one data processor (not shown) produces one or more real-time, virtual indicium based on the calculated orientation and position to guide the physician, surgeon, or surgical team in orienting or positioning the eye. The at least one marking 710 may consist of, but is not limited to, boxes, circles, lines, or checkerboard-type patterns.

In one exemplary embodiment, handle 720 may be attached to stabilization ring 700. Handle 720 can be used by a surgeon to hold stabilization ring 700 on the surface of an eye.

In another embodiment stabilization ring 700 has one or more small levels 730 attached. For example, the horizontal and vertical readings of levels 730 are designed to be identified by at least one data processor (not shown) when they appear in at least one real-time multidimensional visualization. In turn, the at least one data processor can generate data including the level of stabilization ring 700 to be indicated on the display. In some embodiments, the at least one data processor (not shown) can produce one or more real-time, virtual indicium based on the calculated level to guide the physician, surgeon, or surgical team in leveling the eye.

In one embodiment, at least one groove 740 is made into the upper surface of stabilization ring 700. A surgeon can use at least one groove 740 to direct an inserter with an implant into an eye. Stabilization ring 700 may also have at least one additional marking 760 indicating the angle of at least one groove 740.

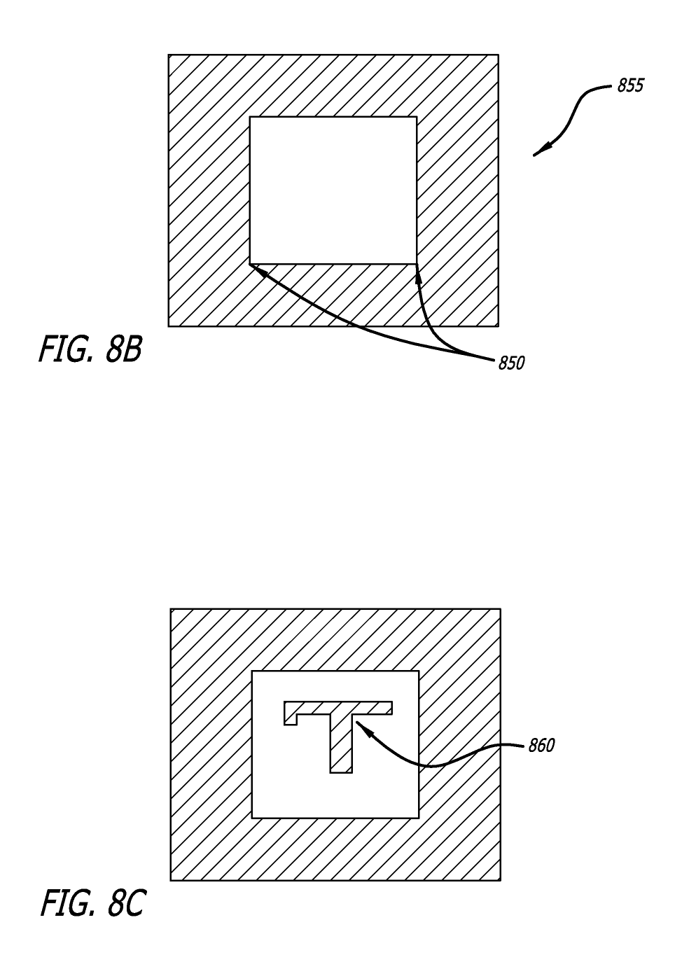

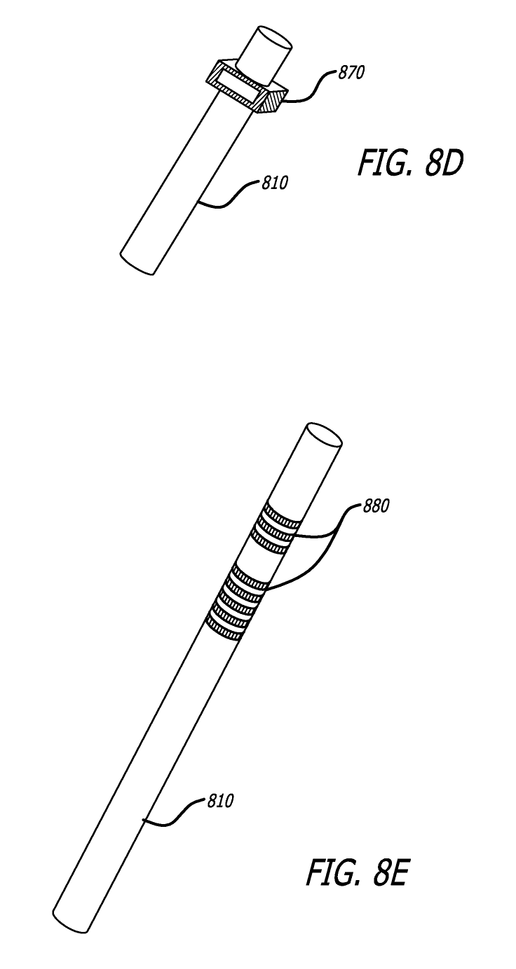

FIG. 8A is an illustration of an exemplary embodiment of inserter 800. Inserter tip 850 of inserter 800 can be used by a surgeon to guide implant 840 into the anterior chamber of an eye. Implant 840 can be attached to inserter 800 by a variety of different methods. For example without limitation, implant 840 may be press-fit inside inserter 800, and inserter 800 can have a plunger (not shown) that can be depressed by the surgeon to release implant 840 once properly placed in an eye. In another exemplary embodiment, inserter 800 can have a latching mechanism whereby the surgeon can release implant 840 by pressing a button to retract a catch between inserter tip 850 and implant 840. At least one marking 820 can be laser etched, painted, drawn, molded, or along the surface of inserter handle 810. Alternatively, at least one marking 820 can be indicated by LEDs emitting either visible or non-visible wavelengths, such as, but not limited to infrared LEDs. At least one marking 820 is designed to be identified by at least one data processor (not shown) when it appears in at least one real-time multidimensional visualization. As the surgeon guides inserter 800 into an eye, the at least one data processor (not shown) can use disparity between the visualization images of at least one marking 820 to calculate the position, orientation, and/or angle of inserter 800 and generate this data for display. In some embodiments, the at least one data processor (not shown) can produce one or more real-time, virtual indicium based on the calculated position, orientation, and/or angle to guide the physician, surgeon, or surgical team in moving the inserter into the eye. In some embodiments, the generated real-time, virtual indicia can include a real-time virtual implant and/or a real-time virtual inserter tip based on the position, orientation, and/or angle calculated from the at least one marking on the inserter as identified in the at least one real-time multidimensional visualization.

In one embodiment of inserter 800, inserter handle 810 can also include at least one length or depth measurement marking 830 laser etched, painted, drawn, molded, or indicated by LEDs along the surface of inserter handle 810. At least one length or depth measurement marking 830 can be in millimeters. This at least one length or depth measurement marking 830 is designed to be identified by least one data processor (not shown) when it appears in at least one real-time multidimensional visualization. In turn, in some embodiments, the at least one data processor can calculate the depth of inserter 800 as the surgeon guides it into an eye and generate this data for display. In one embodiment, the at least one data processor (not shown) can produce one or more real-time, virtual indicium based on the calculated depth to guide the physician, surgeon, or surgical team in the implant procedure.

The at least one data processor can calculate the position, orientation, angle, and/or depth of inserter 800 relative to stabilization ring 700, relative to the eye, and/or . relative to a microscope or gonioscope.

FIG. 8B is an illustration of a high contrast marking 855 that can be laser etched, painted, drawn, or molded along the surface of an inserter handle or laser etched, painted, drawn, or molded on a flat plane that can be wrapped around the inserter handle. High contrast markings are useful for accurately calculating position, orientation and/or depth. For example, corners 850 of the high contrast nested boxes in FIG. 8B can be calculated to sub-pixel accuracy.

FIG. 8C is an illustration of an exemplary rotationally and axially asymmetric marking 860 that can be laser etched, painted, drawn, or molded along the surface of an inserter handle or laser etched, painted, drawn, or molded on a flat plane that can be wrapped around the inserter handle. Rotationally and axially asymmetric markings are useful for calculating position, orientation and/or depth of an inserter or stabilization ring in accordance with the teachings of the present disclosure.

FIG. 8D an illustration of an exemplary embodiment of at least one marking on at least one flat plane 870 wrapped around inserter handle 810. Markings can be laser etched, painted, drawn, or molded on at least one flat plane 870 and at least one flat plane 870 can be wrapped around inserter handle 810. At least one marking on at least one flat plane 870 is designed to be identified by at least one data processor (not shown) when it appears in at least one real-time multidimensional visualization. In one embodiment, at least three flat planes with at least one marking are wrapped around the inserter handle. In other embodiments, up to eight flat planes are wrapped around the inserter handle.

FIG. 8E is an illustration of an exemplary markings 880 laser etched, painted, drawn, or molded along the surface of inserter handle 810.

As a first step in a pressure-relieving implant procedure according to the present description, a pre-operative data set can be captured or obtained. The pre-operative data set can include any portion of data about a patient including, for example, the patient's weight, age, hair color, intraocular pressure, bodily features, medical history, and at least one image of at least a portion of the patient's target surgical anatomy, specifically the eye, even more specifically, information about the iridocorneal angle. In some embodiments, pre-operative data can be identified through optical coherence tomography (OCT) imaging.

In an exemplary embodiment, the pre-operative dataset, or pre-operative patient data includes a still image of at least a portion of the eye, particularly the iridocorneal angle, of the patient undergoing glaucoma surgery. In some embodiments, the pre-operative still image is in HD. A pre-operative data set can also include a mark-up of the patient's eye for analysis, measurement, or alignment as well as topographical data or measurements.

In one embodiment, wherein a pre-operative data set is collected, a slit lamp microscope is used to collect the data. A “slit lamp” is an instrument commonly consisting of a high intensity light source that can be adapted to focus and shine the light as a slit. A slit lamp allows an optometrist or ocular surgeon to view parts of the eye in greater detail than can be attained by the naked eye. Thus, a slit lamp can be used to view the cornea, retina, iris and sclera of a patient's eye. A conventional slit lamp can be retro-fitted with an image capture module as described herein, preferably with at least one photosensor. This allows a surgeon or optometrist to comfortably collect accurate and reliable pre-operative patient data including at least one still image of the patient's eye, preferably under natural dilation and most preferably in HD.

In a second step, the pre-operative data set still image, or just still image, captured in the first step is matched to a real-time multidimensional visualization of at least a portion of the target surgical field. Matching the still image to the multidimensional visualization is important because the target surgical field may have changed since the pre-operative image still was captured such as by tissue shifting and rotating when the patient changes position. As a result, the measurements obtained during the pre-operative examination may no longer be accurate or easily aligned in light of such changes in the patient's physical alignment and position. Additionally, any surgical markings that may have been applied to the patient's tissues during the pre-operative examination may have shifted, been wiped away, or blurred.

At this point, the pre-operative still image of the patient's eye is analyzed by a surgeon, a surgical team or the at least one data processor of the apparatus to identify at least one distinct visible feature that is static and recognizable relative to and within the still image of the eye. Utilizing the teachings described herein, this at least one distinct visible feature is used to align the image with the real-time multidimensional visualization of the target surgical field during the actual surgery. Preferably, this real-time visualization is a 3D HD visualization of the target surgical field.

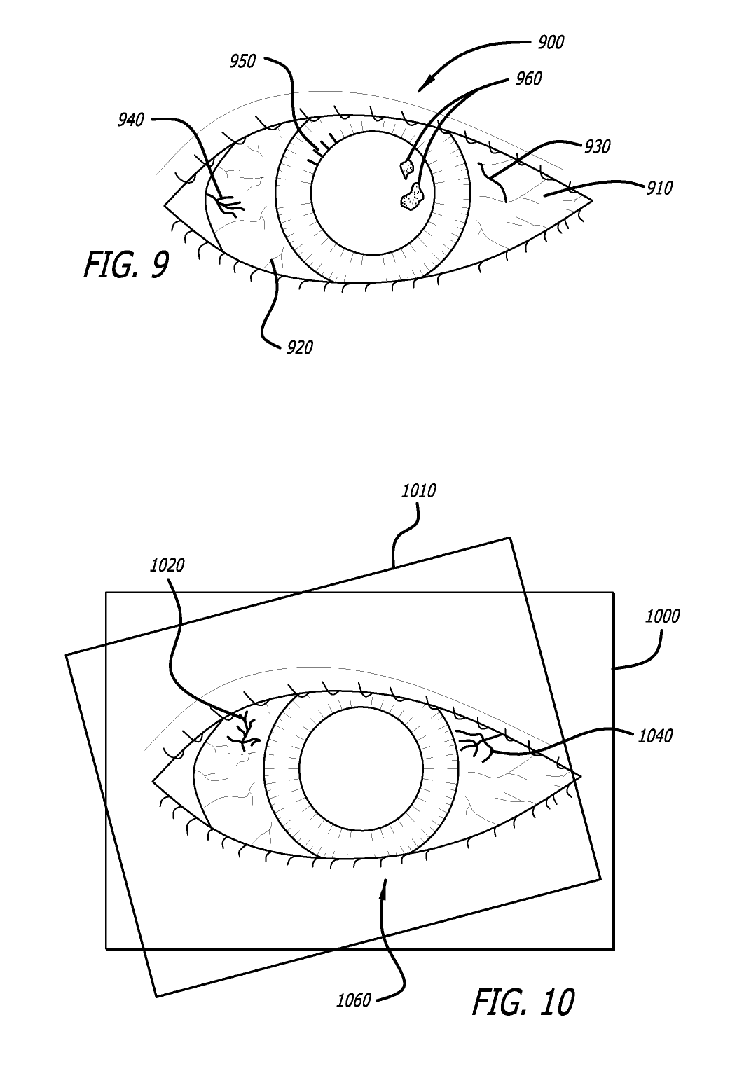

For example, referring to FIG. 9, one or more exemplary distinct visible features that can be identified are illustrated in sclera 910 of eye 900. However, recognizable visible features can also be identified within the iris, on the cornea, or on the retina of the eye. Exemplary distinct visible features include, without limitation, surface vasculature 920, visible vascular networks 930 and vascular branching patterns 940, iris patterns 950, scratches on the cornea, dimples on the cornea, retinal features 960, deformities, voids, blotches, sequestered pigment cells, scars, darker regions, and combinations thereof. Additionally, both the pupillary boundary and limbus are distinct visible features, either of which can be utilized in accordance with the teachings of the present description to align and track the image in conjunction with the real-time visualization of the target surgical field.

In one embodiment, once at least one distinct visible feature has been identified in the pre-operative patient data still image, the still image and the associated visible feature or features are stored for later processing and use in the operating room. It should be noted that the pre-operative patient data need not be taken in a separate operation or at a separate location from the operating room or theater. For example, during surgery to repair a traumatic injury or to simplify a patient's visit, the entire process can be performed in the operating room to save time.

A third step involves the surgeon, the surgical team, the at least one data processor, or a combination thereof aligning or registering the pre-operative still image of the target surgical anatomy or field with the real-time multidimensional visualization of the target surgical field. Generally speaking, this alignment is accomplished utilizing specific static visual features identified within the pre-operative still image of the target surgical site to align the still image with the real-time multidimensional visualization of the target surgical field. This allows the pre-operative image to be aligned accurately with the tissues of the target surgical field regardless of whether the target surgical field has shifted, rotated or reoriented relative to other patient tissues or structures following collection of the pre-operative data.

The pre-operative still image of the patient's eye is overlaid on one or more real-time 3D HD visualizations of at least a portion of the patient's target surgical field for at least a portion of the surgical procedure. Referring to FIG. 10, exemplary real-time 3D HD visualization 1000 of a patient's eye is overlaid with pre-operative patient data still image 1010 of the same eye. Previously identified and recognizable distinct vascular networks in the sclera of the patient's eye, identified on the left as reference numeral 1020 and on the right as reference numeral 1040 of eye 1060 are used to align pre-operative patient data still image 1010 with real-time 3D HD visualization 1000.

In one embodiment, the pre-operative data may consist of OCT image slices of the anterior chamber of an eye. A OCT dataset will contain features similar to a still image, which can be used for aligning or registering the pre-operative data with the real-time multidimensional visualization of the target surgical field. The features may include, but are not limited to: blood vessels, moles, lesions, scars, limbus and iris boundaries, iris colorations and/or cell growth anomalies.

Once a still image or OCT dataset has been properly aligned or registered either by a surgeon, a surgical team, at least one data processor or a combination thereof, the surgeon can lock the image or data in place. Because a patient undergoing ocular surgery is not under general anesthesia, the eye or target surgical field may be moving or rotating during surgery. In some embodiments, a snapshot of the real-time multidimensional surgical visualization may be used to facilitate alignment or registration of the pre-operative data with the real-time multidimensional visualization of the target surgical field.

In an optional fourth calibration step, the controlling surgeon places a calibration target having known dimensions and features into the real-time multidimensional visualization of the target surgical field and triggers the apparatus to calibrate the target surgical field into consistent and useful measurable dimensions.

In a further step, the at least one data processor produces at least one real-time virtual indicium or multiple real-time virtual indicia including data for guiding at least one implant to a desired angle, a desired depth, and/or a desired position in an eye for display on the real-time visualization of the target surgical field. The virtual indicia including data for guiding at least one implant into an eye can be highly patient specific.

In some embodiments, the indicia including data for guiding at least one implant into an eye can include pre-determined shapes, such as, but not limited to, arcs, lines, circles, ellipses, squares, rectangles, trapezoids, diamonds, triangles, polygons and irregular volumes including specific information pertaining to the angle, depth, and position at which the implant should be inserted. In some embodiments the real-time, virtual indicia can include a virtual implant or a virtual inserter tip used for guiding an implant into an eye. Such indicia can be generated based on the actual position of an inserter with at least one marking or a stabilization ring with at least one marking. The virtual implant and/or virtual inserter tip can have utility because the iridocorneal angle is usually obscured from a surgeon's view by the sclera. Thus, as the inserter and implant pass out of view, the virtual indicia can be used to illustrate their position, orientation, and/or depth beneath the sclera, iris, or other opaque tissue. In other embodiments real-time virtual indicia can include a virtual stabilization ring in reference to which the inserter can be used to guide an implant into the anterior chamber of an eye.

It is also within the scope of the present disclosure that a surgeon may input one or more freehand virtual indicia on a still image or real-time multidimensional visualization. Additionally, it is also contemplated as being within the scope of the present description to utilize pre-operative markings that are placed within the target surgical field on the patient so that the data processor will generate virtual surgical indicia including data guiding at least one implant to a desired angle, a desired depth, and/or a desired position in an eye according to the markings found on the pre-operative data set or on the patients themselves.

Further still, a surgeon may utilize multiple different virtual indicia including data for guiding at least one implant to a desired angle, a desired depth, and/or a desired position in an eye during a single surgical procedure or any subpart thereof. For example, initial virtual indicia including data for guiding at least one implant to a desired angle, a desired depth, and/or a desired position in an eye may be replaced by other indicia including data for guiding at least one implant to a desired angle, a desired depth, and/or a desired position in an eye at any point during a surgery, or two or more different indicia may be used to represent more complex surgical markings. In some embodiments, virtual indicia in the form of a virtual implant or virtual inserter tip can be continually replaced and updated in real time.

Even further still, the at least one virtual indicia including data for guiding at least one implant to a desired angle, a desired depth, and/or a desired position in an eye can be tailored to a surgeons particular needs. Data for the desired depth, desired angle, desired orientation and/or desired position of the implant will be based on both inputted data and algorithms used by the surgeon to generate them. The algorithms used by the surgeon can be tailored or can be replaced by any appropriate re-calculated algorithm known to those of ordinary skill in the art.

It should also be noted that when desired to correspond to a real-time 3D HD visualization of the target surgical field, the real-time virtual surgical indicia including data for guiding at least one implant to a desired angle, a desired depth, and/or a desired position in an eye, and in some embodiments including a virtual implant or a virtual inserter tip, can be generated in 3D as well as in HD, or both, depending on the particular surgical procedure or upon the needs of the surgeon. In some embodiments, either the real-time virtual indicia or data for guiding at least one implant to a desired angle, a desired depth, and/or a desired position in an eye can be in 3D and/or HD and vice versa. For example, and not intended to be a limitation, a 3D HD real-time virtual indicia can be paired with 2D standard definition data for guiding at least one implant to a desired angle, a desired depth, and/or a desired position in an eye.

It should be noted that it is within the scope and teachings of the present disclosure that the virtual indicia including data for guiding at least one implant into an eye, and in some embodiments including a virtual implant or a virtual inserter tip, can be sized and modified according to the needs of the surgeon. For example, the indicium including data for guiding at least one implant into an eye can be sized, rotated and moved horizontally, vertically, and in depth as needed by the surgeon.

Further, the virtual indicia including data for guiding at least one implant into an eye, and in some embodiments including a virtual implant, a virtual inserter tip and/or a virtual stabilization ring, can be composed of different types of indication markings and can be in HD. For example, without limitation, the markings can be monochromatic or colored, with varying levels of transparency, composed of thin or thick lines, dashed or solid lines, a series of different shapes and the like as is consistent with contemporary digital graphics technology. Further, the graphic presentation can be different within individual indicia to more easily visualize the indicium in different areas or to emphasize specific areas of interest.

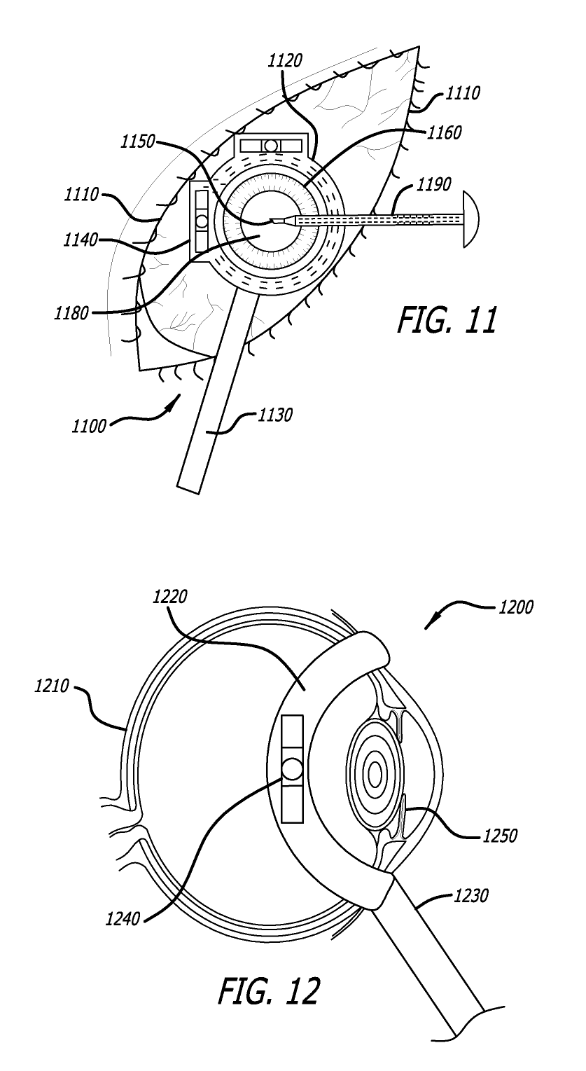

FIG. 11 is a plan view of an exemplary embodiment of a stabilization ring and an exemplary embodiment of an inserter in use on eye 1100. Stabilization ring 1120 is held in place by handle 1130 on cornea 1180 between eyelids 1110. Levels 1140 attached can be attached to stabilization ring 1120 in order to assist a surgeon to level and center stabilization ring 1120 on cornea 1180. Iris 1160 of eye 1100 is visible beneath stabilization ring 1120. Inserter 1190 is used by a surgeon to guide implant 1150 into the anterior chamber between cornea 1180 and iris 1160.

FIG. 12 depicts the cross-section of an eye with an exemplary embodiment of a stabilization ring placed on the eye's surface. Stabilization ring 1220 is held on the surface of eye 1210 so that iris 1250 is visible beneath the stabilization ring 1220. One embodiment of stabilization ring 1220 includes handle 1230 to hold stabilization ring 1220 in place. Another example includes levels 1240 so that the surgeon can ensure stabilization ring 1220 is centered and level on eye 1210.

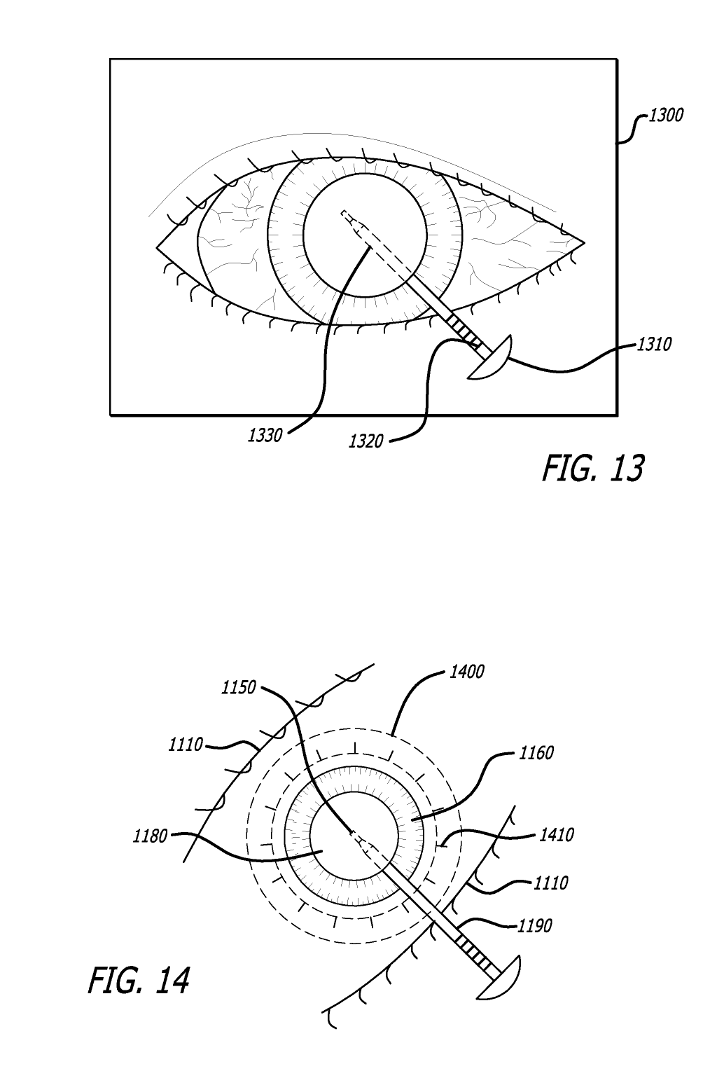

FIG. 13 is a front view of an exemplary embodiment of a real-time 3D HD visualization of an eye 1300 including generated real-time, virtual indicia. As the inserter 1310 moves into the eye, at least one data processor calculates the position, orientation, and/or angle of inserter 1310 based on the location of at least one marking 1320. In some embodiments, the at least one data processor then generates real-time virtual implant and/or a real-time virtual inserter tip 1330 on the display. Generated real-time virtual implant and/or a real-time virtual inserter tip 1330 can assist a surgeon in guiding an implant to a precise location within the anterior chamber of an eye. Other generated display data can include images, numbers, tables, or script indicating the orientation, position, or level of the eye or stabilization ring, position, orientation, or angle of the inserter, or depth of the inserter.

FIG. 14 is a plan view of an exemplary embodiment of a generated real-time virtual stabilization ring 1400 on eye. At least one data processor can generate real-time virtual stabilization ring 1400 on cornea 1180 between eyelids 1110 such that iris 1160 is visible. In some embodiments, real-time virtual stabilization ring 1400 can have at least one virtual marking 1410 that can be used to guide inserter 1190 with implant 1150 into the anterior chamber of an eye. In some embodiments, at least one virtual marking 1410 can include angle and/or level.

A further understanding of the present disclosure will be provided to those of ordinary skill in the art from an analysis of exemplary steps utilizing the apparatus described above to practice the associated methods disclosed herein. The apparatus and methods of the present description provides a surgeon with the ability to create and use one or more user adjustable, accurate, real-time, virtual indicium including data for guiding at least one shunt, stent, valve, or drain to a desired depth, a desired angle, and/or a desired position within the anterior chamber of an eye.

A surgeon will find that the apparatus and methods disclosed herein provide many advantages over existing technology. Firstly, as ocular surgeons are aware, markings commonly associated with guiding at least one implant to a desired angle, a desired depth, and/or a desired position in an eye are hard to estimate with the naked eye, and even if markings are made on the eye itself, those markings are not commonly effective once a procedure has commenced. The present disclosure provides apparatus and methods which assist a surgeon in guiding at least one implant to a desired angle, a desired depth, and/or a desired position in an eye by providing easy to see real-time virtual indicia that is determined pre-operatively and compared to the current location, depth, position, or angle of the stabilization ring and inserter on the eye.

Further, the virtual reference indicium or indicia including data for guiding at least one implant into an eye are not affected by the surgical procedure itself. Therefore, they remain as constant references even when the target tissues are subjected to fluids and wiping. More importantly, the indicia including data for guiding at least one implant to a desired angle, a desired depth, and/or a desired position within an eye are precise and tissue and structure specific, rather than the approximations known to those of ordinary skill in the art. Further, the indicium can be changed, removed, and reinstated as needed to provide an added degree of control and flexibility to the performance of a surgical procedure. For example, a controlling surgeon can chose to vary the transparency or remove a reference indicium including guiding at least one implant into an eye altogether from a visualization to give a clearer view of underlying tissues or structural features and then reinstate the indicium to function as a template or guide the implant procedure.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the exemplary embodiments (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the exemplary embodiments and does not pose a limitation on the scope of the exemplary embodiments otherwise claimed. No language in the specification should be construed as indicating any non-claimed element essential to the practice of the exemplary embodiments.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the exemplary embodiments. Of course, variations on these described embodiments will become apparent to those of ordinary skill in the art upon reading the foregoing description. The inventor expects skilled artisans to employ such variations as appropriate, and the inventors intend for the embodiments to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Furthermore, numerous references have been made to patents and printed publications. Each of the above-cited references is individually incorporated herein by reference in their entirety.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Exemplary embodiments so claimed are inherently or expressly described and enabled herein.

In closing, it is to be understood that the exemplary embodiments disclosed herein are illustrative of the principles of the present disclosure. Other modifications that may be employed are within the scope of the disclosure. Thus, by way of example, but not of limitation, alternative configurations of the present exemplary embodiments may be utilized in accordance with the teachings herein. Accordingly, the present exemplary embodiments are not limited to that precisely as shown and described.

We claim:

1. A system for guiding at least one implant into an eye, said system comprising:
at least one real-time, multidimensional visualization module producing a real-time multidimensional visualization at least a portion of which is presented on at least one display;
at least one data processor configured to produce at least one virtual indicium including data for guiding said at least one implant to a desired angle, a desired depth, a desired position or a combination thereof in said eye in conjunction with said at least one real-time multidimensional visualization of at least a portion of said eye; and,
at least one inserter guiding said implant to said desired depth, said desired angle, and/or said desired position in said eye in conjunction with said data processor and said real-time multidimensional visualization module.

2. A system according to claim 1, further comprising at least one stabilization ring configured to level, fixate, and/or orient said eye in conjunction with said data processor and said real-time multidimensional visualization module.

3. A system according to claim 2 wherein said at least one stabilization ring is virtual and incorporated into said at least one virtual indicium produced by said at least one data processor.

4. A system according to claim 1 wherein said at least one virtual indicium is at least a portion of a real-time virtual implant or a real-time virtual inserter tip.

5. A system according to claim 1 wherein said at least one data processor includes an input for receiving pre-operative patient data to produce said data for guiding said implant to a desired angle, depth, and/or position.

6. A system according to claim 5 wherein said pre-operative patient data comprises at least one pre-operative stereoscopic still image, at least one pre-operative optical coherence tomography image, or a combination of both.

7. A system according to claim 6 wherein said at least one pre-operative stereoscopic still image or said at least one pre-operative optical coherence tomography image includes at least one specific visual feature identifiable by a surgeon wherein said at least one specific visual feature includes vasculature, vascular networks, vascular branching patterns, patterns in the iris, scratches on the cornea, dimples on the cornea, retinal features, the limbus, the pupillary boundary, deformities, voids, blotches, sequestered pigment cells, scars, darker regions, and combinations thereof.

8. A system according to claim 1 wherein said at least one real-time, multidimensional visualization is three dimensional (3D) and/or in high definition (HD).

9. A system according to claim 1 wherein said at least one implant is a shunt, a stent, a drain, or a valve.

10. A system according to claim 2 wherein said at least one stabilization ring has at least one marking, wherein said at least one marking is a laser etched, painted, drawn, molded, or light emitting diode pattern and said at least one marking is identifiable by said at least one data processor wherein said at least one data processor utilizes said at least one marking in calculating the position and/or level of said eye.

11. A system according to claim 2 wherein said at least one stabilization ring has at least one groove that directs said at least one inserter into said eye and at least one marking that indicates the angle of said at least one groove.

12. The system according to claim 2 wherein said at least one stabilization ring has a handle for guiding, placing, or holding said at least one stabilization ring on said eye.

13. A system according to claim 1 wherein said at least one inserter has at least one marking identifiable by said at least one data processor wherein said at least one data processor utilizes said at least one marking in calculating angle, depth, orientation, and/or position of said at least one inserter within said eye.

14. A stabilization ring comprising a surface between the inner diameter and the outer diameter of a ring, wherein said surface has at least one marking and is utilized to level, fixate, and/or orient an eye and wherein said ring is sized to fit an eye.

15. A stabilization ring according to claim 14 wherein said surface is substantially flat.

16. A stabilization ring according to claim 14 wherein said surface is raised.

17. A stabilization ring according to claim 14 wherein said surface is curved.

18. A stabilization ring according to claim 14 wherein said stabilization ring is disposable.

19. A stabilization ring according to claim 14 wherein said at least one marking is a laser etched, painted, drawn, molded, or light emitting diode pattern.

20. A stabilization ring according to claim 14 wherein surface has at least one groove and at least one marking that indicates the angle of said groove, wherein said groove is utilized for directing an inserter into an eye.

21. A stabilization ring according to claim 14 wherein said stabilization ring has a handle attached for guiding, placing, and/or holding said stabilization ring on the surface of said eye.

22. A stabilization ring according to claim 14 wherein said stabilization ring has at least one level, wherein said at least one level is utilized to level said stabilization ring on said eye.

23. An inserter comprising a handle with at least one marking wherein said at least one marking is a laser etched, painted, drawn, molded or light emitting pattern and said at least one marking is utilized for calculating depth, orientation and/or position of said inserter within an eye.

24. An inserter according to claim 22 wherein said inserter is disposable.

25. An inserter according to claim 22 for use with a system for guiding at least one implant into an anterior chamber of an eye, said system comprising:
at least one real-time, multidimensional visualization module producing a real-time multidimensional visualization of at least a portion of at least one display;
at least one data processor configured to produce at least one virtual indicium including data for guiding said at least one implant to a desired angle, a desired depth, and/or a desired position in said eye in conjunction with said at least one real-time multidimensional visualization of at least a portion of said eye and said disposable inserter; and
at least one stabilization ring configured to level, fixate, and/or orient said eye in conjunction with said data processor and said real-time multidimensional visualization module.
